(19) United States
(12) Patent Application Publication
(10) Pub. No.: US 2006/0045900 A1
(43) Pub. Date: Mar. 2, 2006

(54) Embolization

(76) Inventors: Robert Richard, Wrentham, MA (US); Thomas V. Casey II, Grafton, MA (US)

Correspondence Address: FISH & RICHARDSON PC, P.O. BOX 1022, MINNEAPOLIS, MN, 55440-1022, US

(21) Appl. No.: 10/927,868

(22) Filed: Aug. 27, 2004

(51) Int. Cl. A61K 31/74

(52) U.S. Cl. 424/423

(57) ABSTRACT

Embolization and related methods are disclosed.

### TECHNICAL FIELD

The invention relates to embolization, as well as related methods.

### BACKGROUND

Therapeutic vascular occlusions (embolizations) are used to prevent or treat pathological conditions in situ. Embolic compositions (e.g., liquid embolic compositions, compositions including embolic particles) are used for occluding vessels in a variety of medical applications. In some instances, a gel is used to occlude a vessel.

### SUMMARY

In one aspect, the invention features a method that includes disposing a polymer into a device that can deliver the polymer into a lumen of a subject. The method also includes interacting the polymer with a gelling agent to form a gel. The polymer is a sulfonated polymer, a carboxylated polymer, or a phosphated polymer.

In another aspect, the invention features a method that includes disposing a polymer into a device that can deliver the polymer into a lumen of a subject, delivering the polymer into the lumen of the subject, adding a gelling agent into the lumen of the subject, and interacting the gelling agent with the polymer to form a gel. The polymer is a sulfonated polymer, a carboxylated polymer, or a phosphated polymer.

In an additional aspect, the invention features a method that includes interacting a polymer with a gelling agent to form a gel. The polymer is associated with a therapeutic agent, and the interaction of the polymer with the gelling agent releases the therapeutic agent from the polymer.

In a further aspect, the invention features a method that includes interacting a gelling agent with a polymer to form a gel. The gelling agent is associated with a therapeutic agent, and the interaction of the gelling agent with the polymer releases the therapeutic agent from the gelling agent.

In another aspect, the invention features a method that includes disposing a polymer in a liquid into a device that can deliver the polymer into a lumen of a subject. The method also includes changing the pH of the liquid to gel the polymer. The polymer is a sulfonated polymer, a carboxylated polymer, or a phosphated polymer.

Embodiments can include one or more of the following features.

The method can include interacting the polymer with the gelling agent within, and/or outside of, a device that is configured to deliver the polymer into a lumen of a subject. The gel that is formed by the interaction between the polymer and the gelling agent can then be delivered into the lumen of the subject. The gel can be delivered into the lumen by, for example, percutaneous injection. Alternatively or additionally, a gel can be formed within a lumen of a subject by interacting the polymer with the gelling agent within the lumen. In embodiments in which the gel has been formed within, or delivered into, the lumen of a subject, the method can further include embolizing the lumen of the subject with the gel. The method can further include shaping the gel within the lumen of the subject. In certain embodiments, the method can further include converting the gel into a non-gel form (e.g., a liquid form), for example, after embolization. In some embodiments in which the gel is converted into a non-gel form, the gel can be present in the lumen of the subject before being converted into the non-gel form.

The method can further include incorporating a therapeutic agent into the polymer and/or gelling agent prior to contacting the polymer with the gelling agent. In embodiments in which the therapeutic agent is incorporated into the polymer and/or gelling agent prior to contacting the polymer with the gelling agent, the therapeutic agent can be released from the polymer and/or gelling agent as they contact and form a gel. In some embodiments, the method can further include incorporating a therapeutic agent into the gel. The therapeutic agent can be released from the gel, for example, into the lumen of a subject.

The polymer can be a block copolymer, such as a block copolymer that includes a styrene monomer, and/or an isobutylene monomer, and/or an ethylene monomer, and/or a butylene monomer. For example, the polymer can be sulfonated styrene-isobutylene-styrene or sulfonated styrene-ethylene/butylene-styrene.

In some embodiments, the polymer can be disposed in a liquid in the device, and the gelling agent can change the pH of the liquid to gel the polymer.

The polymer can include a therapeutic agent prior to contacting the gelling agent. In some embodiments in which the polymer includes a therapeutic agent, the therapeutic agent can be released from the polymer by interacting the polymer with the gelling agent, and/or by an ion exchange reaction.

In certain embodiments, the gelling agent can include a salt, such as calcium chloride or sodium chloride. In some embodiments, the gelling agent can include an organic molecule with a functional group. For example, the gelling agent can be polyvinylpyridine, or can include an organic molecule with a functional group that includes an amine, such as diamine-terminated polyethylene oxide. The gelling agent can be cationic. For example, the gelling agent can include an ammonium ion (e.g., an alkyl ammonium ion).

In certain embodiments, the gel can be dimensioned to fit within the lumen of the subject. In some embodiments (e.g., embodiments in which the gel is dimensioned to fit within the lumen of the subject), the gel can have a maximum dimension of from about 1000 microns to about 2500 microns.

The gel can be used to embolize the lumen of the subject, and/or to treat a cancer condition. For example, the gel can embolize a lumen that is associated with a cancer condition.

In some embodiments, the device can be configured to fit within the lumen of the subject. The device can include, for example, a catheter, a syringe, or a cannula (e.g., a syringe or a cannula with at least two chambers).

Embodiments can include one or more of the following advantages.

In some embodiments, a gel can be formed in situ (i.e., inside the subject). For example, the gel can be formed inside a lumen of a vessel of a subject (e.g., inside a lumen of a vessel to be embolized, such as an artery of a human). This can, for example, reduce or eliminate the cost and/or complexity associated with storing and/or handling an embolic material. In general, the particular location of gel formation can be selected as desired. For example, the conditions can be selected so that the gel forms at or near a target site inside the lumen of the vessel of the subject. This can, for example, enhance the flexibility associated with an embolization procedure, and/or allow for the use of a relatively small delivery device.

In certain embodiments, a gel can be formed under conditions that result in the gel having dimensions corresponding to the environment of the gel. For example, in embodiments in which a gel is formed inside a lumen of a vessel of a subject, the gel can be dimensioned to occlude the lumen (e.g., an artery of a human). This can, for example, reduce the cost and/or complexity associated with delivering an embolic material to a target site inside a subject.

In some embodiments, a gel can be converted into a non-gel form, such as a liquid form. Thus, for example, if the gel is accidentally formed at the wrong location, then the gel can be converted into a liquid and dispersed, allowing another gel to be formed in its place. A gel that can be converted into a non-gel form can also be used, for example, in a temporary embolization procedure. After the procedure is over, the gel can be converted into, for example, a liquid form, and dispersed from the embolization site.

In general, the components that are used to form a gel are in liquid form (e.g., in the form of a solution). This allows the components to exhibit relatively good deliverability to a desired location (e.g., the target site inside the lumen of the subject).

Features and advantages are in the description, drawings, and claims.

### DESCRIPTION OF DRAWINGS

FIG. 1 is a side view of the proximal end portion of an embodiment of a device, as the device is being used in an embolization procedure.

FIG. 2 is a side view of the distal end portion of the device of FIG. 1.

### DETAILED DESCRIPTION

In general, a gel can be formed at or near a target site. The gel can be formed from components (e.g., liquid components) that can be more easily delivered to the target site than the gel itself would be. Once formed, the gel can exhibit good occlusive properties because, for example, the gel can be tailored to fit the size and/or shape of the target site.

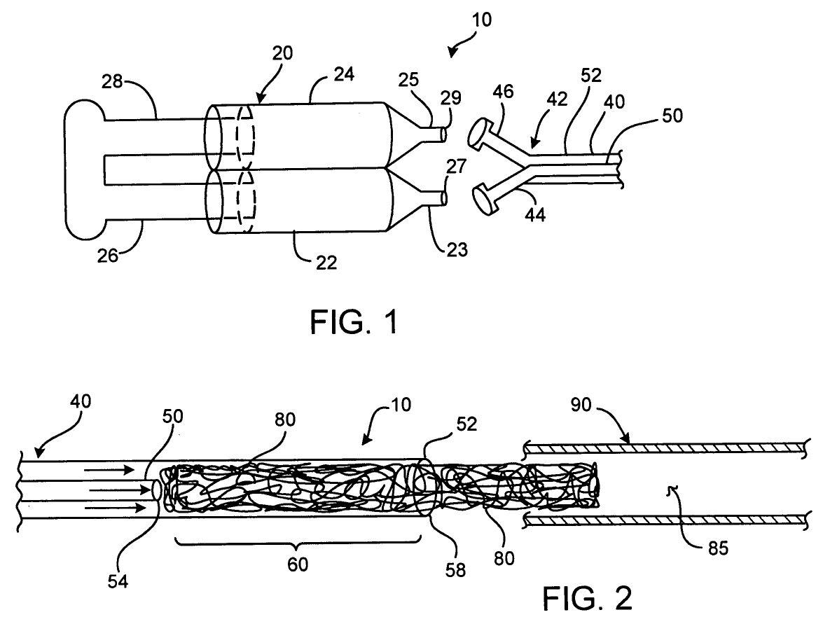

FIGS. 1 and 2 show a delivery device 10 including a double-barrel syringe 20 and a cannula 40 that are capable of being coupled such that substances contained within syringe 20 are introduced into cannula 40. Syringe 20 includes a first barrel 22 having a tip 23 with a discharge opening 27, and a second barrel 24 having a tip 25 with a discharge opening 29. Syringe 20 further includes a first plunger 26 that is movable in first barrel 22, and a second plunger 28 that is movable in second barrel 24. First barrel 22 contains a gelling agent-containing liquid (e.g., calcium chloride in a solvent, such as water or a biocompatible alcohol), while second barrel 24 contains a polymer-containing liquid (e.g., a sulfonated styrene-isobutylene-styrene (“SIBS”) polymer and a solvent, such as water or a biocompatible alcohol). In its proximal end portion, cannula 40 includes an adapter 42 with a first branch 44 that can connect with tip 23, and a second branch 46 that can connect with tip 25. First branch 44 is integral with a first tubular portion 50 of cannula 40, and second branch 46 is integral with a second tubular portion 52 of cannula 40. First tubular portion 50 is disposed within second tubular portion 52. Delivery devices are described, for example, in Sahatjian et al., U.S. Pat. No. 6,629,947, which is incorporated herein by reference.

When cannula 40 is connected to syringe 20 and plungers 26 and 28 are depressed, the sulfonated SIBS-containing liquid moves from second barrel 24 into second tubular portion 52, and the calcium chloride-containing liquid moves from first barrel 22 into first tubular portion 50. The sulfonated SIBS-containing liquid exits first tubular portion 50 and contacts the calcium chloride-containing liquid in a mixing section 60 of second tubular portion 52. The sulfonated SIBS-containing liquid and the calcium chloride-containing liquid interact to form a gel (e.g., a biocompatible gel) 80 within mixing section 60. Gel 80 exits delivery device 10 at a distal end 58 of mixing section 60, and is delivered into a lumen 85 of a vessel 90 of a subject (e.g., an artery of a human) where gel 80 can embolize lumen 85.

Without wishing to be bound by theory, it is believed that gel 80 forms as a result of ionic interactions between calcium ions from the calcium chloride-containing liquid and sulfonate groups from the sulfonated SIBS-containing liquid. It is believed that the ionic interactions cause salts to form, and that the formation of these salts allows the sulfonated SIBS to gel by collapsing or folding together. For example, in embodiments in which the sulfonated SIBS has multiple sulfonate groups along its backbone, it is believed that before interacting with the calcium ions the sulfonate groups can repel each other, causing the polymer to adopt a relatively straight configuration. It is further believed that interaction between the calcium ions and the sulfonate groups forms salts, decreasing the repulsion between the sulfonate groups and allowing the polymer to fold together and turn into a gel. It is also believed that, in some embodiments, the use of SIBS as a polymer can enhance the elastomeric properties and/or deformability of the gel. It is believed that this may be due to the presence of the styrenic portion of the copolymer.

In general, the size and shape of gel 80 can be selected as desired. For example, gel 80 can have dimensions that correspond to the environment in which it is formed (e.g., lumen 85). In other words, as gel 80 is formed, it can fill lumen 85 and assume the shape of lumen 85, such that the dimensions of gel 80 correspond to the dimensions of lumen 85. This can allow gel 80 to effectively occlude lumen 85. In some embodiments, gel 80 can have a maximum dimension of from about 1000 microns to about 2500 microns (e.g., from about 1200 microns to about 1500 microns). Alternatively or additionally, gel 80 can have a minimum dimension of from about 10 microns to about 200 microns (e.g., from about 50 microns to about 150 microns).

Typically, the density of gel 80 is selected to effect occlusion at a target site. In some embodiments, gel 80 can have a density of from about one gram per cubic centimeter to about five grams per cubic centimeter (e.g., from about one gram per cubic centimeter to about 1.5 grams per cubic centimeter). In certain embodiments, as the concentration of polymer in a polymer-containing liquid that is used to form a gel increases, the density of the gel that is formed also increases.

Gel 80 can be used in any of a number of different embolic applications. Gel 80 can be formed at and/or delivered to various sites in the body, including, for example, sites having cancerous lesions, such as the breast, prostate, lung, thyroid, or ovaries. Gel 80 can be used in, for example, neural, pulmonary, and/or AAA (abdominal aortic aneurysm) applications. Gel 80 can be used in the treatment of, for example, fibroids, tumors, internal bleeding, arteriovenous malformations (AVMs), and/or hypervascular tumors. Gel 80 can be used as, for example, fillers for aneurysm sacs, AAA sac (Type II endoleaks), endoleak sealants, arterial sealants, and/or puncture sealants, and/or can be used to provide occlusion of other lumens such as fallopian tubes. Fibroids can include uterine fibroids which grow within the uterine wall (intramural type), on the outside of the uterus (subserosal type), inside the uterine cavity (submucosal type), between the layers of broad ligament supporting the uterus (interligamentous type), attached to another organ (parasitic type), or on a mushroom-like stalk (pedunculated type). Internal bleeding includes gastrointestinal, urinary, renal and varicose bleeding. AVMs are, for example, abnormal collections of blood vessels (e.g. in the brain) which shunt blood from a high pressure artery to a low pressure vein, resulting in hypoxia and malnutrition of those regions from which the blood is diverted. In some embodiments, gel 80 can be used to prophylactically treat a condition.

In certain embodiments, the formation of gel 80 can result in the release of a therapeutic agent (e.g., a drug) into lumen 85. For example, the sulfonate groups in the sulfonated SIBS can be ionically bonded to a therapeutic agent. When the sulfonated SIBS-containing liquid and the calcium chloride-containing liquid interact, the calcium ions from the calcium chloride can participate in an ion-exchange reaction with the therapeutic agent. During the ion-exchange reaction, the sulfonate groups can release the therapeutic agent, and ionically bond to the calcium ions.

In general, a therapeutic agent can be negatively charged, positively charged, amphoteric, or neutral. Examples of therapeutic agents include materials that are biologically active to treat physiological conditions; pharmaceutically active compounds; gene therapies; nucleic acids with and without carrier vectors; oligonucleotides; gene/vector systems; DNA chimeras; compacting agents (e.g., DNA compacting agents); viruses; polymers; hyaluronic acid; proteins (e.g., enzymes such as ribozymes); immunologic species; nonsteroidal anti-inflammatory medications; oral contraceptives; progestins; gonadotrophin-releasing hormone agonists; chemotherapeutic agents; and radioactive species (e.g., radioisotopes, radioactive molecules). Non-limiting examples of therapeutic agents include anti-thrombogenic agents; antioxidants; angiogenic and anti-angiogenic agents and factors; calcium entry blockers; and survival genes which protect against cell death.

Examples of non-genetic therapeutic agents include: (a) anti-thrombotic agents, such as heparin, heparin derivatives, urokinase, and PPack (dextrophenylalanine proline arginine chloromethylketone); (b) anti-inflammatory agents, such as dexamethasone, prednisolone, corticosterone, budesonide, estrogen, sulfasalazine and mesalamine; (c) anti-neoplastic/antiproliferative/anti-mitotic agents, such as paclitaxel, 5-fluorouracil, cisplatin, vinblastine, vincristine, epothilones, endostatin, angiostatin, angiopeptin, agents (e.g., monoclonal antibodies) capable of blocking smooth muscle cell proliferation, and thymidine kinase inhibitors; (d) anesthetic agents, such as lidocaine, bupivacaine and ropivacaine; (e) anti-coagulants, such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters, such as growth factors, transcriptional activators, and translational promoters; (g) vascular cell growth inhibitors, such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents, such as triclosan, cephalosporins, aminoglycosides and nitrofurantoin; (m) cytotoxic agents, cytostatic agents, and cell proliferation affectors; (n) vasodilating agents; (o) agents that interfere with endogenous vasoactive mechanisms; (p) inhibitors of leukocyte recruitment, such as monoclonal antibodies; (q) cytokines and (r) hormones.

Examples of genetic therapeutic agents include anti-sense DNA and RNA, as well as DNA coding for: (a) anti-sense RNA; (b) tRNA or rRNA to replace defective or deficient endogenous molecules; (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor; (d) cell cycle inhibitors including CD inhibitors; and (e) thymidine kinase (“TK”) and other agents useful for interfering with cell proliferation. Other examples of genetic therapeutic agents include DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include plasmids, viral vectors, such as adenoviruses (AV), gutted adenoviruses, adeno-associated virus (AAV), retroviruses, alpha virus (e.g., Semliki Forest, Sindbis, etc.), lentiviruses, herpes simplex virus, replication competent viruses (e.g., ONYX-015) and hybrid vectors; and non-viral vectors, such as artificial chromosomes and mini-chromosomes, plasmid DNA vectors (e.g., pCOR), cationic polymers (e.g., polyethyleneimine (PEI)), graft copolymers (e.g., polyether-PEI and polyethylene oxide-PEI), neutral polymers (e.g., PVP, SP1017 (available from SUPRATEK)), lipids (e.g., cationic lipids), liposomes, lipoplexes, nanoparticles, and microparticles, with or without targeting sequences such as the protein transduction domain (PTD).

Cells include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, and skeletal myocytes or macrophages. Other examples of cells include cells from animal, bacterial or fungal sources (xenogeneic). The cells can be genetically engineered, if desired (e.g., to deliver proteins of interest).

Examples of therapeutic agents are disclosed in, for example, Kunz et al., U.S. Pat. No. 5,733,925, which is incorporated herein by reference. Therapeutic agents disclosed in this patent include the following: “Cytostatic agents” (i.e., agents that prevent or delay cell division in proliferating cells, for example, by inhibiting replication of DNA or by inhibiting spindle fiber formation). Representative examples of cytostatic agents include modified toxins, methotrexate, adriamycin, radionuclides (e.g., such as disclosed in Fritzberg et al., U.S. Pat. No. 4,897,255), protein kinase inhibitors, including staurosporin, a protein kinase C inhibitor of the following formula:

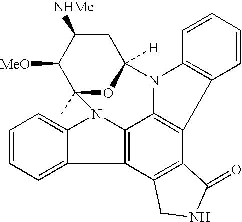

as well as diindoloalkaloids having one of the following general structures:

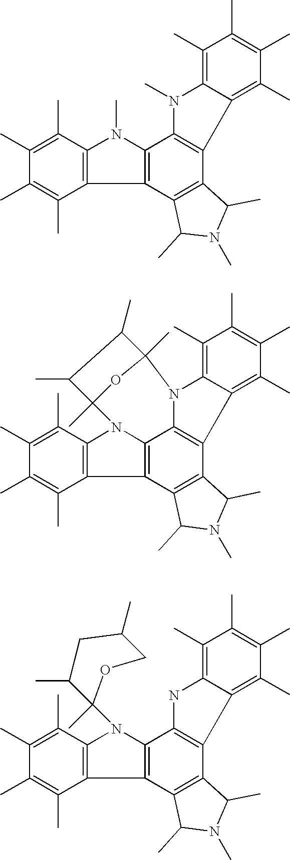

as well as stimulators of the production or activation of TGF-beta, including Tamoxifen and derivatives of functional equivalents (e.g., plasmin, heparin, compounds capable of reducing the level or inactivating the lipoprotein Lp(a) or the glycoprotein apolipoprotein(a)) thereof, TGF-beta or functional equivalents, derivatives or analogs thereof, suramin, nitric oxide releasing compounds (e.g., nitroglycerin) or analogs or functional equivalents thereof, paclitaxel or analogs thereof (e.g., taxotere), inhibitors of specific enzymes (such as the nuclear enzyme DNA topoisomerase II and DNA polymerase, RNA polymerase, adenyl guanyl cyclase), superoxide dismutase inhibitors, terminal deoxynucleotidyl-transferase, reverse transcriptase, antisense oligonucleotides that suppress smooth muscle cell proliferation and the like.

Other examples of “cytostatic agents” include peptidic or mimetic inhibitors (i.e., antagonists, agonists, or competitive or non-competitive inhibitors) of cellular factors that may (e.g., in the presence of extracellular matrix) trigger proliferation of smooth muscle cells or pericytes: e.g., cytokines (e.g., interleukins such as IL-1), growth factors (e.g., PDGF, TGF-alpha or -beta, tumor necrosis factor, smooth muscle- and endothelial-derived growth factors, i.e., endothelin, FGF), homing receptors (e.g., for platelets or leukocytes), and extracellular matrix receptors (e.g., integrins). Representative examples of useful therapeutic agents in this category of cytostatic agents addressing smooth muscle proliferation include: subfragments of heparin, triazolopyrimidine (trapidil; a PDGF antagonist), lovastatin, and prostaglandins E1 or I2.

Agents that inhibit the intracellular increase in cell volume (i.e., the tissue volume occupied by a cell) such as cytoskeletal inhibitors or metabolic inhibitors. Representative examples of cytoskeletal inhibitors include colchicine, vinblastin, cytochalasins, paclitaxel and the like, which act on microtubule and microfilament networks within a cell. Representative examples of metabolic inhibitors include staurosporin, trichothecenes, and modified diphtheria and ricin toxins, Pseudomonas exotoxin and the like. Trichothecenes include simple trichothecenes (i.e., those that have only a central sesquiterpenoid structure) and macrocyclic trichothecenes (i.e., those that have an additional macrocyclic ring), e.g., a verrucarins or roridins, including Verrucarin A, Verrucarin B, Verrucarin J (Satratoxin C), Roridin A, Roridin C, Roridin D, Roridin E (Satratoxin D), Roridin H.

Agents acting as an inhibitor that blocks cellular protein synthesis and/or secretion or organization of extracellular matrix (i.e., an “anti-matrix agent”). Representative examples of “anti-matrix agents” include inhibitors (i.e., agonists and antagonists and competitive and non-competitive inhibitors) of matrix synthesis, secretion and assembly, organizational cross-linking (e.g., transglutaminases cross-linking collagen), and matrix remodeling (e.g., following wound healing). A representative example of a useful therapeutic agent in this category of anti-matrix agents is colchicine, an inhibitor of secretion of extracellular matrix. Another example is tamoxifen for which evidence exists regarding its capability to organize and/or stabilize as well as diminish smooth muscle cell proliferation following angioplasty. The organization or stabilization may stem from the blockage of vascular smooth muscle cell maturation in to a pathologically proliferating form.

Agents that are cytotoxic to cells, particularly cancer cells. Preferred agents are Roridin A, Pseudomonas exotoxin and the like or analogs or functional equivalents thereof. A plethora of such therapeutic agents, including radioisotopes and the like, have been identified and are known in the art. In addition, protocols for the identification of cytotoxic moieties are known and employed routinely in the art.

Other examples of therapeutic agents include therapeutic agents that can be used, for example, in vascular treatment regimens (e.g., as agents targeting restenosis), such as: (a) Ca-channel blockers including benzothiazapines (e.g., diltiazem, clentiazem), dihydropyridines (e.g., nifedipine, amlodipine, nicardapine), and phenylalkylamines (e.g., verapamil); (b) serotonin pathway modulators including 5-HT antagonists (e.g., ketanserin, naftidrofuryl), as well as 5-HT uptake inhibitors (e.g., fluoxetine); (c) cyclic nucleotide pathway agents including phosphodiesterase inhibitors (e.g., cilostazole, dipyridamole), adenylate/Guanylate cyclase stimulants such as forskolin, as well as adenosine analogs; (d) catecholamine modulators including α-antagonists (e.g., prazosin, bunazosine), β-antagonists (e.g., propranolol), and α/β-antagonists (e.g., labetalol, carvedilol); (e) endothelin receptor antagonists; (f) nitric oxide donors/releasing molecules including organic nitrates/nitrites (e.g., nitroglycerin, isosorbide dinitrate, amyl nitrite), inorganic nitroso compounds such as sodium nitroprusside, sydnonimines (e.g., molsidomine, linsidomine), nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine; (g) ACE inhibitors (e.g., cilazapril, fosinopril, enalapril); (h) ATII-receptor antagonists (e.g., saralasin, losartin); (i) platelet adhesion inhibitors (e.g., albumin, polyethylene oxide); (j) platelet aggregation inhibitors including aspirin and thienopyridine (e.g., ticlopidine, clopidogrel) and GP IIb/IIIa inhibitors (e.g., abciximab, epitifibatide, tirofiban); (k) coagulation pathway modulators including heparinoids (e.g., heparin, low molecular weight heparin, dextran sulfate, β-cyclodextrin tetradecasulfate), thrombin inhibitors (e.g., hirudin, hirulog, PPACK(D-phe-L-propyl-L-arg-chloromethylketone), argatroban), FXa inhibitors (e.g., antistatin, TAP (tick anticoagulant peptide)), Vitamin K inhibitors such as warfarin, as well as activated protein C; (l) cyclooxygenase pathway inhibitors (e.g., aspirin, ibuprofen, flurbiprofen, indomethacin, sulfinpyrazone); (m) natural and synthetic corticosteroids (e.g., dexamethasone, prednisolone, methprednisolone, hydrocortisone); (n) lipoxygenase pathway inhibitors (e.g., nordihydroguairetic acid, caffeic acid); (o) leukotriene receptor antagonists; (p) antagonists of E- and P-selectins; (q) inhibitors of VCAM-1 and ICAM-1 interactions; (r) prostaglandins and analogs thereof including prostaglandins such as PGE1 and PGI2 and prostacyclin analogs (e.g., ciprostene, epoprostenol, carbacyclin, iloprost, beraprost); (s) macrophage activation preventers including bisphosphonates; (t) HMG-CoA reductase inhibitors (e.g., lovastatin, pravastatin, fluvastatin, simvastatin, cerivastatin); (u) fish oils and omega-3-fatty acids; (v) free-radical scavengers/antioxidants (e.g., probucol, vitamins C and E, ebselen, trans-retinoic acid, SOD mimics); (w) agents affecting various growth factors including FGF pathway agents such as bFGF antibodies and chimeric fusion proteins, PDGF receptor antagonists (e.g., trapidil), IGF pathway agents including somatostatin analogs (e.g., angiopeptin, ocreotide), TGF-β pathway agents such as polyanionic agents (e.g., heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators (e.g., sulotroban, vapiprost, dazoxiben, ridogrel), as well as protein tyrosine kinase inhibitors (e.g., tyrphostin, genistein, and quinoxaline derivatives); (x) MMP pathway inhibitors (e.g., marimastat, ilomastat, metastat); (y) cell motility inhibitors such as cytochalasin B; (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine, 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, paclitaxel, epothilone), caspase activators, proteasome inhibitors, angiogenesis inhibitors (e.g., endostatin, angiostatin, squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Therapeutic agents are described, for example, in Pinchuk et al., U.S. Pat. No. 6,545,097, and in co-pending U.S. Patent Application Publication No. US 2004/0076582 A1, published on Apr. 22, 2004, both of which are incorporated herein by reference.

While certain embodiments have been described, other embodiments are possible.

As an example, while embodiments have been described in which a sulfonated SIBS-containing liquid is used to form a gel, other styrenic block copolymers may also be used to form a gel. In some embodiments, the styrenic portion of the copolymer can result in a gel that is somewhat elastomeric and/or deformable. Examples of styrenic block copolymers include block copolymers which have at least one styrene monomer, isobutylene monomer, ethylene monomer, and/or butylene monomer. As an example, the polymer can include a styrenic block copolymer such as styrene-ethylene/butylene-styrene (“SEBS”). Such polymers are commercially available as the Kraton® G family of polymers, available from Kraton® Polymers. In certain embodiments, the polymer can be a sulfonated non-styrenic copolymer, such as polyethylene sulfonic acid, sulfonated polyethylene terephthalate, or sulfonated polyphosphazene. In some embodiments, the hardness or softness of a gel formed by contacting a polymer-containing liquid with a gelling agent-containing liquid can be affected by the type of polymer that is used in the polymer-containing liquid. For example, in certain embodiments, the polymer-containing liquid can include a thermoplastic elastomer, such as SIBS, that is selected to form a gel with a particular hardness or softness. The hardness or softness of the gel that forms may depend on the relative proportion of hard blocks and soft blocks within the thermoplastic elastomer. For example, as the ratio of polystyrene (hard) blocks to polyisobutylene (soft) blocks in SIBS increases, the hardness of the gel that forms can also increase. Block copolymers are described, for example, in Pinchuk et al., U.S. Pat. No. 6,545,097, incorporated supra.

Additional examples of polymers that can be used in the formation of a gel include polyvinyl alcohols, polyacrylic acids, polymethacrylic acids, poly vinyl sulfonates, carboxymethyl celluloses, hydroxyethyl celluloses, substituted celluloses, polyacrylamides, polyethylene glycols, polyamides (e.g., nylon), polyureas, polyurethanes, polyesters, polyethers, polystyrenes, polysaccharides (e.g. alginate), polylactic acids, polyethylenes, polymethylmethacrylates, polyethylacrylate, polycaprolactones, polyglycolic acids, poly(lactic-co-glycolic) acids (e.g., poly(d-lactic-co-glycolic) acids), and copolymers or mixtures thereof. In some embodiments, the polymer can be a highly water insoluble, high molecular weight polymer. An example of such a polymer is a high molecular weight polyvinyl alcohol (PVA) that has been acetalized. The polymer can be substantially pure intrachain 1,3-acetalized PVA and substantially free of animal derived residue such as collagen. In general, the polymers are biocompatible.

In certain embodiments, the polymer can be a bioabsorbable polymer (e.g., a polysaccharide, such as alginate).

Generally, the polymer includes one or more functional groups. The functional groups can be negatively charged or positively charged, and/or can be ionically bonded to the polymer. In some embodiments, the functional groups can enhance the biocompatibility of the polymer. Alternatively or additionally, the functional groups can enhance the clot-forming capabilities of the polymer. Examples of functional groups include phosphate groups, carboxylate groups, sulfonate groups, sulfate groups, phosphonate groups, and phenolate groups. For example, a polymer can be a sulfonated styrenic polymer. While sulfonated SIBS has been described above, another example of a suitable sulfonated styrenic polymer is sulfonated SEBS. Generally, as the number of sulfonate groups on a polymer increases, the water solubility of the polymer increases. Thus, a highly sulfonated polymer may also be highly water-soluble (i.e., the polymer may be dissolved in water to form a polymer-containing liquid). Sulfonation of styrene block copolymers is disclosed, for example, in Ehrenberg, et al., U.S. Pat. No. 5,468,574; Vachon et al., U.S. Pat. No. 6,306,419; and Berlowitz-Tarrant, et al., U.S. Pat. No. 5,840,387, all of which are incorporated herein by reference. Examples of other functionalized polymers include phosphated SIBS, phosphated SEBS, carboxylated SIBS, and carboxylated SEBS. In certain embodiments, a polymer can include more than one different type of functional group. For example, a polymer can include both a sulfonate group and a phosphate group.

In some embodiments, more than one polymer can be used to form the gel.

As another example, while embodiments have been described in which calcium chloride is used as a gelling agent, other gelling agents may also be used. Generally, the gelling agents are biocompatible. The gelling agent can be, for example, an ion (e.g., an anion, a cation) and/or a salt (e.g., an inorganic salts) that has either monovalent or multivalent cations. As an example, a suitable gelling agent is a salt with a divalent cation that can ionically cross-link with the polymer. Examples of salts include alkali metal salts, alkaline earth metal salts, and transition metal salts. In some embodiments, a gelling agent can be a calcium, barium, zinc or magnesium salt. In embodiments in which the polymer is alginate, a suitable gelling agent for the gelling agent-containing liquid can once again be calcium chloride. The calcium cations in the calcium chloride gelling agent have an affinity for the carboxyl groups in alginate, and can complex with the carboxyl groups. Another example of a gelling agent is sodium chloride. In embodiments in which the gelling agent is an ion, the ion can be inorganic or organic. For example, the ion can be an ammonium ion (e.g., an alkyl ammonium ion).

In some embodiments, the gelling agent can be an organic molecule with one or more functional groups (a functionalized organic molecule). The functional group in the organic molecule can be, for example, an amine-containing functional group (e.g., a monoamine, a diamine, a triamine). Examples of amine-containing functional groups include amino acids, such as arginine. Examples of functionalized organic molecules include diamine-terminated polyethylene oxide and polyvinylpyridine.

In some embodiments, more than one gelling agent can be used to form the gel.

As an additional example, in some embodiments, a gelling agent can cause a polymer (e.g., sulfonated SIBS, sulfonated SEBS) to gel by forming one or more cross-link bridges between different sections of the polymer. The cross-link bridges can pull the different sections of the polymer closer together, thereby causing the polymer to gel. For example, in certain embodiments, a gelling agent that includes a multivalent cation (e.g., a gelling agent that includes calcium or zinc, such as calcium chloride) can cause a polymer to gel by forming cross-link bridges between different sections of the polymer.

As another example, while ionic interactions have been described, in some embodiments, a gelling agent (e.g., a multiisocyanate) can covalently bond with a polymer (e.g., a polyalcohol polymer, such as polyvinyl alcohol), and can thereby cause the polymer to form a gel.

In some embodiments, a gelling agent can cause a polymer to gel by altering the environment of the polymer. As an example, a gelling agent can alter the pH of the environment surrounding a polymer, which, for example, can cause a neutral polymer to become charged, or can cause a charged polymer to become neutral or more or less charged. The change in pH can thus change the form and/or solubility of the polymer, which can cause the polymer to gel. Examples of gelling agents that may alter the pH of the environment surrounding a polymer and cause the polymer to gel include acetic acid and polyacids (e.g., acrylic acid). Examples of polymers that may be caused to gel by a change in pH include sulfonated SIBS, sulfonated SEBS, carboxylated SIBS, carboxylated SEBS, phosphated SIBS, and phosphated SEBS. As another example, a gelling agent in the gelling agent-containing liquid can be relatively incompatible with a polymer in the polymer-containing liquid. When the gelling agent interacts with the polymer, it can repel the polymer and cause the polymer to gel (e.g., by folding in on itself). Examples of such gelling agents include alcohols such as isopropyl alcohol and ethyl alcohol, and examples of corresponding polymers include sulfonated SEBS and sulfonated SIBS.

As another example, while embodiments have been described in which one or more therapeutic agents are released during formation of the gel, in some embodiments a therapeutic agent alternatively or additionally is contained within the gel. In certain embodiments, the therapeutic agent can be physically bound within the gel. For example, a gel can encapsulate one or more therapeutic agents. A therapeutic agent can become encapsulated in a gel as the gel forms. For example, if the therapeutic agent is released from a polymer during an ion-exchange reaction in which the polymer binds to a gelling agent to form a gel, some or all of the therapeutic agent may become physically entrapped within the gel as the gel forms. The configuration and/or composition of a gel that encapsulates a therapeutic agent can affect delivery of the therapeutic agent from the gel. As an example, a gel that includes a polymer such as sulfonated SIBS, through which water can diffuse, can release a therapeutic agent via diffusion. As another example, the rate of diffusion of a therapeutic agent out of a gel that has a relatively low density may be higher than the rate of diffusion of the same therapeutic agent out of a gel that has a relatively high density. In some embodiments, as the amount of gelling agent that is reacted with a polymer during formation of a gel increases, the density of the gel that forms also increases. In certain embodiments, the therapeutic agent can be chemically bound to one or more components of the gel (e.g., chemically bound to the polymer, chemically bound to the gelling agent). In some embodiments, the therapeutic agent can be physically bound within the gel and chemically bound to one or more components of the gel. In certain embodiments, the polymer-containing liquid can include one or more therapeutic agents, and the gelling agent-containing liquid can contain one or more therapeutic agents (which may be the same as, or different from, the therapeutic agent(s) contained in the polymer-containing liquid).

As an additional example, in some embodiments, a gel that is formed by one of the above-described processes can be converted into a non-gel form, such as a liquid form (e.g., after the gel has been used in an embolization procedure). For example, in some embodiments in which a divalent cation (e.g., a calcium cation) has been used to form a gel by forming cross-link bridges between different sections of a soluble polymer, a monovalent cation (e.g., a sodium cation) can be used to ion-exchange with the calcium cation, thereby undoing the cross-link bridges and causing the polymer to become soluble. As another example, in certain embodiments, the pH of the environment surrounding a gel can be altered (e.g., decreased) to render the gel soluble. For example, in some embodiments in which a gel has been formed out of sulfonated SIBS or sulfonated SEBS, a biocompatible acid (e.g., an amino acid, acetic acid) can be added to the environment of the gel to render the gel soluble.

As another example, in some embodiments, a gel can be bioabsorbable. As a result, the gel can be formed at a target site, and can later be absorbed and/or excreted by the body of the subject (e.g., patient). In certain embodiments, the majority (e.g., at least about 75 weight percent, at least about 90 weight percent, at least about 95 weight percent) of a gel can be formed of one or more bioabsorbable materials.

As a further example, while certain embodiments of delivery devices have been described, other delivery devices may be used. As an example, the delivery device can contain more than two syringes (e.g., when the gel contains more than one polymer and/or more than one gelling agent, in which case a different syringe can be used to delivery each component of the gel). As another example, the delivery device can have plungers that are separately controlled (e.g., so that the polymer(s) and/or gelling agent(s) can be separately delivered to a desired location).

As another example, while embodiments have been described in which the portion of the delivery device in which the gel forms is outside the lumen of the subject as the gel forms, in some embodiments, the portion of the delivery device in which the gel forms can be present within the lumen of the subject as the gel forms.

As an additional example, in some embodiments, a gel can be shaped after it has been formed. In certain embodiments, the gel can be shaped by a delivery device, such as a catheter. For example, a catheter can have an orifice with an adjustable diameter at its distal end. As a gel that has been formed within the catheter exits the catheter through the orifice, the orifice can be sized to shape the gel as desired. In certain embodiments, a delivery device (e.g., a catheter) can include a chamber at one of its ends, and the gel can be delivered into the chamber, where the gel can conform to the shape of the chamber. Thereafter, the gel can be delivered from the chamber to a target site. In some embodiments, a gel can be mechanically shaped within the lumen of a subject. For example, a gel can be formed within a lumen, and two balloons can be delivered into the lumen, such that a balloon is disposed on either side of the gel. The balloons can then be pushed toward each other, thereby compacting and shaping the gel. In some embodiments, the balloons may alternatively or additionally be used to maintain the gel in a particular location until the gel has fully formed (e.g., until the gel has cured). In certain embodiments, a gel that has formed within a lumen can be shaped and/or moved using a steerable arm (e.g., a steerable arm that is attached to the delivery device).

As another example, while embodiments have been described in which a polymer-containing liquid and a gelling agent-containing liquid interact within a delivery device, in some embodiments a polymer-containing liquid and a gelling agent-containing liquid can interact outside of a delivery device. In certain embodiments, the polymer-containing liquid and the gelling agent-containing liquid can be delivered separately to a target site (e.g., in the lumen of a subject), where they can interact with each other to form a gel that occludes the target site. In such embodiments, prior to and/or during formation of the gel, the target site can be temporarily occluded by, for example, a balloon. This temporary occlusion can provide time for mixing the gelling agent-containing liquid and the polymer-containing liquid to form the gel. The balloon can be removed, for example, once the gel has been formed and is of suitable size to occlude the target site.

As an additional example, in certain embodiments, a gel can be formed outside of a subject using one or more of the methods described above. After the gel has been formed, it can be combined with a carrier fluid (e.g., a saline solution, a contrast agent, or both) to form an embolic composition. The embolic composition can then be disposed in a delivery device (e.g., a syringe, a catheter) and delivered to a target site (e.g., by percutaneous injection). For example, a gel can be formed by contacting calcium chloride with a sulfonated polymer (e.g., sulfonated SIBS), such that calcium cations from the calcium chloride form cross-link bridges on the sulfonated polymer that cause the sulfonated polymer to gel. The resulting gel can be combined with a carrier fluid to form an embolic composition. The embolic composition can then be disposed in, for example, a syringe, and delivered to a target site by percutaneous injection.

As a further example, in some embodiments, a treatment site can be occluded by using one or more of the above-described gels in conjunction with other occlusive devices. In some embodiments, the gels can be used with particles such as those described in Buiser et al., U.S. Published Patent Application No. 2003/0185896 A1, and in U.S. Patent Application Publication No. US 2004/0096662 A1, published on May 20, 2004, both of which are incorporated herein by reference. For example, particles can be delivered to a target site to occlude the target site. Simultaneously or thereafter, a gel can be formed at the target site. The gel can fill the spaces between the particles, and/or can bond the particles to each other. In certain embodiments, the gels can be used in conjunction with one or more particle chains, and/or with one or more coils. Particle chains are described, for example, in U.S. patent application No. 10/830,195, filed on Apr. 22, 2004, and entitled “Embolization”, which is incorporated herein by reference. Coils are described, for example, in Twyford, Jr. et al., U.S. Pat. No. 5,304,195, and Guglielmi et al., U.S. Pat. No. 5,540,680, both of which are incorporated herein by reference.

As another example, in some embodiments in which a gel is used to occlude a target site (e.g., to treat a cerebral aneurysm), an adhesive (e.g., a bioadhesive such as poly(ethylene oxide), carboxymethyl cellulose, or cyanoacrylate) can be injected into the target site so that the adhesive is used in conjunction with the gel. The adhesive can, for example, anchor the gel within the target site.

As a further example, in some embodiments, a gel can be formed near a target site and caused to precipitate into the target site as the gel is formed. For example, a gel can be formed near an aneurysmal sac, such that when the gel is formed, it falls into the aneurysmal sac, filling the sac.

As an additional example, in some embodiments, different gels (e.g., gels having different shapes, sizes, physical properties, and/or chemical properties) can be used together in an embolization procedure. The different gels can be delivered into and/or formed in the body of a subject in a predetermined sequence or simultaneously. In certain embodiments, mixtures of different gels can be delivered using a multi-lumen catheter and/or syringe. In some embodiments, different gels can be capable of interacting synergistically (e.g., by engaging or interlocking) to form a well-packed occlusion, thereby enhancing embolization.

As a further example, in some embodiments the gels can be used for tissue bulking. For example, a gel can be formed in tissue adjacent to a body passageway. The gel can narrow the passageway, thereby providing bulk and allowing the tissue to constrict the passageway more easily. In certain embodiments, a cavity can be formed in the tissue, and the gel can be formed in the cavity. Gel tissue bulking can be used to treat, for example, intrinsic sphincteric deficiency (ISD), vesicoureteral reflux, gastroesophageal reflux disease (GERD), and/or vocal cord paralysis (e.g., to restore glottic competence in cases of paralytic dysphonia). In some embodiments, gel tissue bulking can be used to treat urinary incontinence and/or fecal incontinence. A gel can be used as a graft material or a filler to fill and/or to smooth out soft tissue defects, such as for reconstructive or cosmetic applications (e.g., surgery). Examples of soft tissue defect applications include cleft lips, scars (e.g., depressed scars from chicken pox or acne scars), indentations resulting from liposuction, wrinkles (e.g., glabella frown wrinkles), and soft tissue augmentation of thin lips. Tissue bulking is described, for example, in co-pending Published Patent Application No. US 2003/0233150 A1, published on Dec. 18, 2003, and entitled “Tissue Treatment”, which is incorporated herein by reference.

As another example, while gels that include therapeutic agents have been described, in some embodiments a gel can alternatively or additionally include other materials. For example, in certain embodiments, a gel can include (e.g., encapsulate) a radiopaque material, a material that is visible by magnetic resonance imaging (an MRI-visible material), a ferromagnetic material, and/or an ultrasound contrast agent. Such materials are described, for example, in U.S. Patent Application Publication No. US 2004/0101564, published on May 27, 2004, which is incorporated herein by reference. In certain embodiments, a gel can include a surface preferential material. Surface preferential materials are described, for example, in U.S. patent application No. 10/791,552, filed on Mar. 2, 2004, and entitled “Embolization”, which is incorporated herein by reference.

As an additional example, in certain embodiments, a polymer (e.g., SIBS, polymethylmetacrylate, polyurethane, polyethylacrylate) can be rendered liquid by being dispersed in a surfactant (e.g., sulfonated SIBS, sodium dodecyl sulfate). The surfactant can then be removed and/or destabilized by, for example, contacting the surfactant with a salt (e.g., sodium chloride, calcium chloride) and thereby causing the surfactant to precipitate. Once the surfactant has precipitated, the polymer can form a gel.

Other embodiments are in the claims.

What is claimed is:

1. A method, comprising:
disposing a polymer into a device, the device being configured to deliver the polymer into a lumen of a subject, the polymer being selected from the group consisting of sulfonated polymers, carboxylated polymers, and phosphated polymers; and
interacting the polymer with a gelling agent to form a gel.

2. The method of claim 1, wherein the method includes interacting the polymer with the gelling agent within the device.

3. The method of claim 1, wherein the method includes interacting the polymer with the gelling agent outside of the device.

4. The method of claim 3, wherein the method includes interacting the polymer with the gelling agent within the lumen of the subject.

5. The method of claim 1, further comprising delivering the gel into the lumen of the subject.

6. The method of claim 1, further comprising embolizing the lumen of the subject with the gel.

7. The method of claim 6, wherein the lumen of the subject is associated with a cancer condition.

8. The method of claim 1, wherein the polymer comprises a block copolymer.

9. The method of claim 8, wherein the polymer comprises a styrene monomer.

10. The method of claim 8, wherein the polymer comprises an isobutylene monomer.

11. The method of claim 8, wherein the polymer comprises an ethylene monomer.

12. The method of claim 8, wherein the polymer comprises a butylene monomer.

13. The method of claim 8, wherein the polymer comprises sulfonated styrene-isobutylene-styrene.

14. The method of claim 8, wherein the polymer comprises sulfonated styrene-ethylene/butylene-styrene.

15. The method of claim 1, wherein the gelling agent comprises a salt.

16. The method of claim 1, wherein the gelling agent comprises an organic molecule with a functional group.

17. The method of claim 1, wherein the gelling agent is cationic.

18. The method of claim 1, wherein the polymer is disposed in a liquid in the device and the gelling agent changes the pH of the liquid to gel the polymer.

19. The method of claim 1, further comprising incorporating a therapeutic agent into the gel.

20. The method of claim 19, further comprising releasing the therapeutic agent from the gel into the lumen of the subject.

21. The method of claim 1, further comprising incorporating a therapeutic agent into the polymer prior to contacting the polymer with the gelling agent.

22. The method of claim 1, further comprising incorporating a therapeutic agent into the gelling agent prior to contacting the polymer with the gelling agent.

23. The method of claim 1, wherein the gel is dimensioned to fit within the lumen of the subject.

24. The method of claim 1, wherein a maximum dimension of the gel is from about 1000 microns to about 2500 microns.

25. The method of claim 1, wherein the gel is used to treat a cancer condition.

26. The method of claim 1, wherein the device is configured to fit within the lumen of the subject.

27. The method of claim 1, wherein the device comprises a catheter, a syringe, or a cannula.

28. The method of claim 1, further comprising delivering the gel into the lumen of the subject by percutaneous injection.

29. The method of claim 1, further comprising converting the gel into a non-gel form.

30. The method of claim 29, wherein the gel is present in the lumen of the subject before the gel is converted into the non-gel form.

31. The method of claim 1, further comprising converting the gel into a liquid form.

32. The method of claim 1, further comprising shaping the gel within the lumen of the subject.
